(19) United States
(12) Patent Application Publication
(10) Pub. No.: US 2026/0212499 A1
(43) Pub. Date: Jul. 23, 2026

(54) SYSTEMS AND METHODS FOR GENERATING CUSTOMIZED PROMPTS FOR ZERO-SHOT RARE EVENT MEDICAL IMAGE CLASSIFICATION USING LLM

(71) Applicants: Sandeep Gupta, Phoenix, AZ (US); Payal Kamboj, Tempe, AZ (US); Ayan Banerjee, Gilbert, AZ (US)

(72) Inventors: Sandeep Gupta, Phoenix, AZ (US); Payal Kamboj, Tempe, AZ (US); Ayan Banerjee, Gilbert, AZ (US)

(73) Assignee: Arizona Board of Regents on Behalf of Arizona State University, Tempe, AZ (US)

(21) Appl. No.: 19/450,553

(22) Filed: Jan. 15, 2026

Related U.S. Application Data
(60) Provisional application No. 63/747,856, filed on Jan. 21, 2025.

(51) Int. Cl. G06T 7/00; G06V 10/764; G16H 50/20

(52) U.S. Cl. CPC G06T 7/0012; G06V 10/764; G16H 50/20

(57) ABSTRACT

Example methods and systems incorporate an inventive approach for generating highly accurate and contextually descriptive prompts containing discriminative characteristics are described using domain-specific expert knowledge on rare events to generate customized and contextually relevant prompts, which are then used by large language models (LLMs) for image classification. The zero-shot, privacy-preserving approach enhances rare event classification without additional training, outperforming state-of-the-art techniques.

### CROSS REFERENCE TO RELATED APPLICATIONS

The present document is a Non-Provisional patent application that claims benefit to U.S. Provisional Patent Application Ser. No. 63/747,856 filed on Jan. 21, 2025 which is herein incorporated by reference in its entirety.

### FIELD

The present disclosure generally relates to artificial intelligence including language vision models and medical image analysis; in particular to Al-based methodologies for generating customized prompts for zero-shot rare event medical image classification, among other features described herein.

### BACKGROUND

Rare events are extremely infrequent events and consequently characteristics about such events are highly valuable. Detecting such rare events requires advanced methods that account for their infrequency, unique features, and positive-negative evidence, since the true rare event distribution cannot be fully captured from sparse observations using purely statistical methods. Moreover, due to their infrequent occurrences, rare events do not have much data, and hence deep learning techniques fail in estimating the distribution for such data.

It is with these observations in mind, among others, that various aspects of the present disclosure were conceived and developed.

### SUMMARY

The present disclosure provides a number of examples that describe computer-implemented techniques and operations for zero-shot rare event medical image classification. In the context of the disclosed methods, devices, techniques, apparatus, systems, and so on, the terms “operable to,” “configured to,” and “capable of” used herein are interchangeable.

Disclosed are various non-limiting and example implementations of an inventive concept that enable zero-shot classification of rare events in medical images by combining human expert knowledge with the reasoning capabilities of language models, without requiring additional model training on rare-event data. Raw textual knowledge from sources such as medical literature and clinical studies is refined to extract discriminative, non-overlapping rules for a rare event class. The refined knowledge is structured into knowledge components that can analyze and quantify image-related characteristics. For a given medical image, a knowledge model generates an image-derived textual description and uses the knowledge components to produce rule-based prompt text with a corresponding degree of satisfiability. The rule-based prompt text is integrated into a human knowledge embodied textual prompt (HKETP) and combined with the refined knowledge to form a customized prompt that is then processed by a language model to produce a classification output. In some embodiments, patient privacy is preserved because the language model receives textual prompts rather than the medical image itself.

In one set of illustrative examples, the computer-implemented techniques and operations are embodied by non-transitory computer-readable medium storing instructions that, when executed by one or more processors of a computing system, cause the computing system to: (a) refine raw textual knowledge, using one or more large language models, to generate refined knowledge comprising non-overlapping discriminative knowledge specific to a rare event class; (b) structure the refined knowledge into knowledge components and encode the knowledge components, using at least one of the one or more large language models, to generate rule-based text outputs corresponding to the knowledge components; (c) for an input medical image, generate targeted prompt text for respective ones of the knowledge components, each targeted prompt text having a degree of satisfiability based on the input medical image; (d) integrate the targeted prompt text to generate a human knowledge embodied textual prompt (HKETP); (e) generate a customized prompt for rare event classification by combining the HKETP with at least a portion of the refined knowledge; and (f) cause at least one of the one or more large language models to process the customized prompt, without providing the input medical image to the large language model, to generate a classification output indicating whether the input medical image is associated with the rare event class.

Other illustrative examples are contemplated including computer-readable media, etc. The foregoing examples broadly outline various aspects, features, and technical advantages of examples according to the disclosure in order that the detailed description that follows may be better understood. It is further appreciated that the above operations described in the context of the illustrative example method, device, and computer-readable medium are not required and that one or more operations may be excluded and/or other additional operations discussed herein may be included. Additional features and advantages will be described hereinafter. The conception and specific examples illustrated and described herein may be readily utilized as a basis for modifying or designing other structures for carrying out the same purposes of the present disclosure. Such equivalent constructions do not depart from the spirit and scope of the appended claims.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 is an illustration of an example process flow for generating CUPL (customized language prompts) large language model (LLM)-prompts and image prompts (top), and Customized Prompts via Knowledge and Language models (CuKPL) LLM prompts and classification (bottom).

FIG. 2 is an illustration of generating human knowledge embodied textual prompts (HKETP) and using the LLM-based classification process described herein for inference (to classify the target class from the HKETP as generated and predict the presence of a rare event).

FIG. 3 is a process flow of an example method associated with functionality described herein.

FIG. 4 is a simplified illustration of an example computing device for implementing functionality described herein.

Corresponding reference characters indicate corresponding elements among the view of the drawings. The headings used in the figures do not limit the scope of the claims.

### DETAILED DESCRIPTION

The present disclosure relates to example implementations of an inventive concept for generating descriptive prompts containing discriminative characteristics for zero-shot rare event medical image classification via large language models (LLMs). More specifically, in some examples, the inventive concept includes an open-vocabulary model natural used for image classification including generating highly accurate and contextually descriptive prompts containing discriminative characteristics for rare event detection in the medical field. This can include domain-specific expert knowledge on rare events to generate customized and contextually relevant prompts that can then be used by large language models for image classification.

As indicated herein, technical challenges persist with rare events that, due to their infrequent occurrences, do not have much data, and hence deep learning techniques fail in estimating the distribution for such data. Rare event detection, particularly in critical fields like medicine, is the most challenging due to infrequent occurrence of rare events, low inter-class variability and high intra-class variability. Open-vocabulary models represent an innovative approach to image classification. Unlike traditional models, these models classify images into any set of categories specified with natural language prompts during inference. These prompts usually comprise manually crafted templates (e.g., ‘a photo of a { }’) that are filled in with the names of each category.

The inventive concept is a technical solution that includes a simple yet effective method for generating highly accurate and contextually descriptive prompts containing discriminative characteristics. To address the foregoing technical challenges, the present disclosure includes a novel approach that uses domain-specific expert knowledge on rare events to generate customized and contextually relevant prompts, which can then be used with LLMs for image classification. The zero-shot, privacy-preserving examples of a method described enhance rare event classification without additional training, outperforming state-of-the-art techniques.

Example non-limiting features include the following:
Extraction of human knowledge about rare class event in a structured format with knowledge components.
Combining the knowledge components with human knowledge embodied textual prompts for rare event categories
Inputting the prompt into an LLM
Outputting image classification with patient privacy intact
The privacy of the patient is intact due to the customized prompts and image to text extraction. In some examples, it is intact because patient's image is never shown to the LLM, only the textual prompt describing the image is sent to LLM for the classification

While the inventive concept is primarily described as a method implementation, the inventive concept can take the form of a system, computer/machine readable medium including instructions executable by at least one processing element, and the like.

### 1 Introduction (Including Technical Challenges)

“Rare events are extremely infrequent events whose characteristics make them or their consequences highly valuable. Such events appear with extreme scarcity and are hard to predict, although they are expected eventually” (see Sokolova et al., “Evaluation of rare event detection,” in Advances in Artificial Intelligence: 23rd Canadian Conference on Artificial Intelligence, Canadian Al 2010, Ottawa, Canada, May 31-Jun. 2, 2010. Proceedings 23. Springer, 2010, pp. 379-383.). Despite the rarity, these instances are highly significant as they contain crucial information. Detecting such rare events requires advanced methods that account for their infrequency, unique features, and positive-negative evidence since the true rare event distribution cannot be fully captured from these sparse observations using purely statistical methods. In the current era of large language and vision models, which are extensively trained on public data and demonstrate impressive reasoning capabilities, both pattern recognition and deductive reasoning (i.e., their capacity to follow instructions in reasoning tasks), the present disclosure explores whether these models can be adapted to effectively classify rare events despite the limited availability of event-specific data.

Open-vocabulary models demonstrate high image classification accuracy across various datasets, even without labeled training data specific to those tasks. They achieve this by leveraging extensive collections of image-text pairs from the internet, learning to associate images with their corresponding captions. This method offers significant flexibility during inference. Unlike traditional models, these models classify images by assessing the similarity between an image and a caption. During inference, a caption or ‘prompt’ is generated for each desired category, enabling the model to match each image with the most suitable prompt (standard zero-shot image classification being the process of categorizing images into classes that the model hasn't seen during training). The ability of open-vocabulary models to accurately match text and images stems from the diverse visual and linguistic patterns they have encountered during training, allowing them to generalize well to unseen categories. As a result, they can draw upon these learned associations to align images and text efficiently, even when faced with unfamiliar data. This allows for on-the-fly category selection and adjustments without the need for additional training. However, this innovative approach introduces a new challenge:
How can we effectively describe an image category using natural language prompts for categories that the model has likely never seen before in text or visual?

The conventional method involves manually crafting prompt templates (e.g., “a photo of a { }”), generating a natural language label for each category in the dataset, and creating a collection of prompts for each category by populating each template with the corresponding labels. Subsequently, image embeddings are compared to the nearest set of prompt embeddings and assigned the category linked to that set of prompts. However, these methods have the following drawbacks.

a) The standard prompts, such as “a photo of { }” of standard zero shot lack domain-specific information, which is crucial in medical imaging as public datasets are limited. b) These methods are usually evaluated on datasets where categories have been indirectly encountered during training. For example, a model trained on images of dogs with captions like “Aussie the pup” is likely to classify the category “Dog” correctly during testing due to semantic similarities with the label. In contrast, for rare events, such as seizure onset zones, these models struggle to generalize due to insufficient data and a lack of relevant associations in the training corpus. c) Standard prompt templates lack detailed descriptions which are crucial for fine-grained classification.

To overcome these challenges, the inventive concept includes the proposal of Customized Prompts via Knowledge and Language models (CuKPL), which couples knowledge on rare event from human experts with Large language models (LLM) to enable zero-shot rare event classification. CuKPL utilizes a knowledge model to generate human knowledge embodied textual prompts (HKETP) that are descriptive for rare event categories. In this approach, human knowledge about rare class events is extracted from a thorough review of technical papers and clinical studies, often expressed in vague natural language. This process yields two key outcomes: a) a structured format that encapsulates the relevant insights and findings derived from the literature, and b) knowledge components to analyze and quantify different aspects of knowledge. Integrating these two components give HKETP. During inference, the image is inputted into the knowledge model, which generates HKETP. This prompt is then fed into a LLM, which is tasked with classifying the image based on the provided knowledge.

Rare event detection is challenging in biomedical imaging due to the high intra-class variability, where instances of the same class exhibit different representations, and low interclass variability, where other classes or noise/normal events can produce representations similar to rare events. This is where the discriminative knowledge rules become crucial, helping to differentiate between rare events and similar-looking normal or noisy instances. Extensive experiments demonstrate that CuKPL outperforms state-of-the-art (SOTA) techniques for rare event detection. Operating in a zero-shot framework, CuKPL preserves data privacy as it never sees the image, is energy-efficient and negates the need for synthetic data generation, ineffective in low-data scenarios.

### 2. Related Work

Standard zero-shot using: As discussed herein, standard zero-shot setup uses a predefined template to infer a category without any training. However, these models are trained on large, publicly available datasets, which often lack representation of medical data.

Customized prompts using LLM: A recent approach, CuPL (Pratt et al., “What does a platypus look like? generating customized prompts for zero-shot image classification,” in Proceedings of the IEEE/CVF International Conference on Computer Vision, 2023, pp. 15691-15701.), integrates open-vocabulary models with LLMs to generate customized prompts. CuPL utilizes prompts such as: “What does a {category} look like?” These prompts are made to the LLMs to generate customized prompts based on the LLM's prior knowledge of that category. However, in the medical domain where data is scarce, LLMs struggle to produce detailed and accurate prompts, leading to suboptimal performance for large vision modes (LVMs) in these cases.

SOTA medical imaging classification: SOTA classification methods in medical domain include Vision Transformers (ViT), or 2D-CNNs, which are highly dependent on large datasets. Alternatives have also emerged that leverage domain knowledge alone or in combination with deep learning (DL) models. Combining domain knowledge with Al has shown superior performance compared to standalone methods. However, they are heavily data-reliant.

### 3. Methods

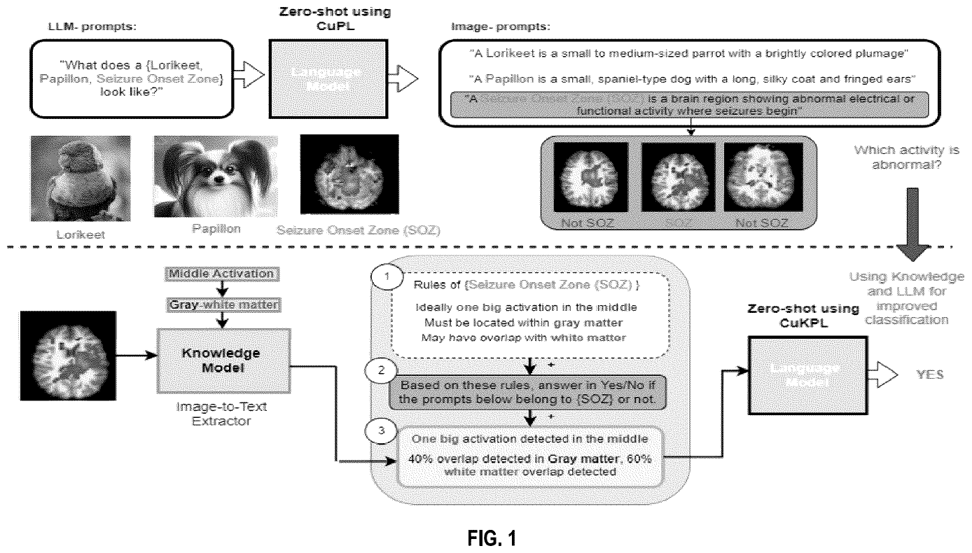

FIG. 1 illustrates an overall example methodology. It extends the SOTA customized prompt driven zero shot image recognition methodology with LVMs like CLIP by incorporating human expert knowledge embodiment in the prompts. In the inference phase, the CuKPL method is presented with an image. CuKPL processes the input image through a knowledge module that generates the HKETP prompt, which can include: i) a generalized prompt containing application-specific knowledge, ii) a query for the LLM to assess prompt consistency with rare class attributes, and iii) human knowledge embodiments specific to the image instance. The LLM then evaluates if the HKETP prompt aligns with rare class characteristics.

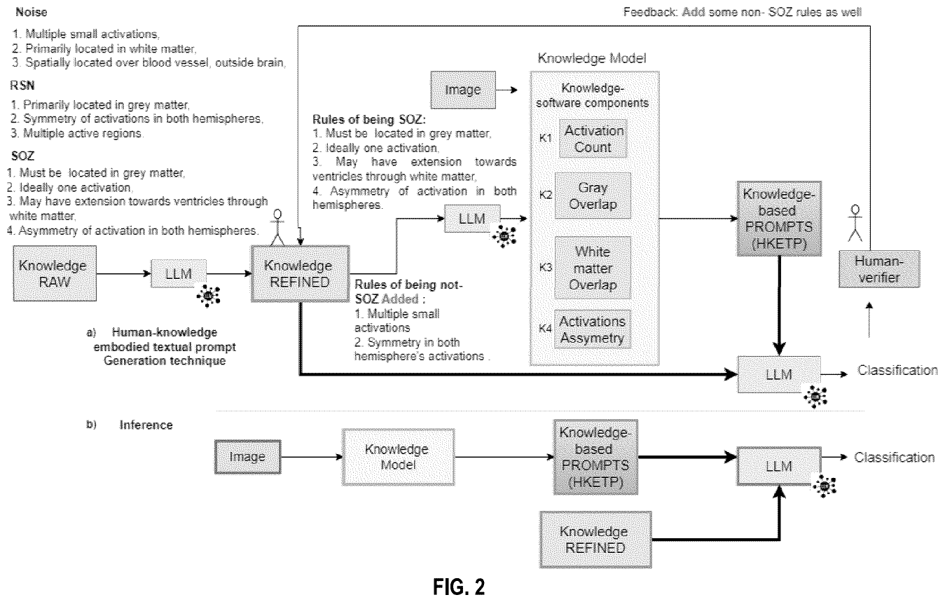

HKETP Generation As illustrated in FIG. 2 (top), raw textual knowledge from various sources, including literature, is initially refined using an LLM to capture non-overlapping, discriminative knowledge specific to the rare class. This refined knowledge is then structured into knowledge components and encoded, with the LLM generating rule-based text outputs. Each knowledge component is encoded by the LLM to produce targeted prompts, each with a degree of satisfiability based on the input image. These generated texts are integrated (FIG. 1), creating HKETP, which, along with the refined knowledge, is then utilized by the LLM for final classification. Following classification, a human verifier provides feedback and updates to the LLM to ensure precise knowledge encoding and adherence to the classification criteria. Inference: FIG. 2 (bottom) illustrates the LLM-based classification process, where the HKETP generated by the knowledge model is combined with the final refined knowledge. The final refined knowledge is the knowledge intended to use for the classification (some examples don't use all available raw knowledge). This final knowledge is achieved through an iterative process involving LLM and a human-verifier. This knowledge is then converted into software functions code by LLM, and each function generates the knowledge relevant statements. The statements from these functions are concatenated (HKETP). The final refined knowledge concatenated with HKETP leads to customized prompt for the rare event classification and is fed to LLM (any LLM) for final classification.

These are utilized by the LLM to classify the target class, predicting the presence of the rare event. This approach enables the LLM to apply deductive reasoning, leveraging encoded fuzzy logic rules and domain-specific knowledge to assess the likelihood of the HKETP belonging to the target class.

In some examples, each knowledge component Ki corresponds to a discriminative characteristic of a rare event class and is encoded as (i) an evaluation procedure configured to determine whether the characteristic is present in an input medical image (or a representation derived therefrom) and (ii) a language output procedure configured to generate a rule-based text output statement describing the characteristic. Each knowledge component may output a degree of satisfiability that indicates whether, or a degree to which, the characteristic is satisfied by the input medical image. The degree of satisfiability may be binary, ordinal, continuous, or fuzzy and may be computed from one or more quantified image-derived measurements, such as a region count, a region size/extent metric, an overlap percentage with an anatomical tissue mask, a spatial asymmetry metric, and/or other quantified measurements derived from the input medical image.

### HKETP Generation and Customized Prompt Structure.

In some embodiments, the system generates targeted prompt text for respective knowledge components based on their satisfiability for the input medical image. The targeted prompt text generated across the knowledge components is integrated to form an HKETP. In some embodiments, a customized prompt used for classification comprises the HKETP combined with at least a portion of refined knowledge, where the refined knowledge provides rule-based criteria for the rare event class and the HKETP provides an instance-specific textual representation of the input medical image derived from the image-derived attributes. The customized prompt may further include one or more instructions directing a language model to output a classification decision based on satisfaction of the criteria.

### Privacy-Preserving Operation.

In some embodiments, patient privacy is preserved by performing image-to-text extraction locally or within a protected computing environment and providing only a textual representation of the input medical image to a language model for classification. The textual representation may include quantified measurements and rule-based statements derived from the image-derived attributes and may exclude pixel data of the input medical image. In some embodiments, the textual representation is de-identified by excluding patient identifiers and removing or redacting protected health information (PHI) when present.

### Human Verifier Feedback and Iterative Refinement.

In some embodiments, a human verifier reviews one or more of: raw knowledge sources, refined knowledge, knowledge component encodings, satisfiability outputs, and/or classification outputs. The human verifier may approve, reject, or modify one or more rules, knowledge components, and/or prompt-generation logic. Based on the feedback, the system updates the refined knowledge and/or knowledge components to improve consistency and adherence to classification criteria. In some embodiments, the system stores updated refined knowledge and knowledge components as versioned artifacts with associated provenance metadata, including at least a source identifier and verification status.

### Conversion to Software Functions.

In some embodiments, at least a portion of the refined knowledge and/or knowledge components is converted, using a language model, into executable software functions. Each software function outputs one or more knowledge-relevant statements based on image-derived attributes and may output a degree of satisfiability. The outputs of the software functions are concatenated or otherwise aggregated to form at least a portion of the HKETP and/or the customized prompt for classification.

### 3.1. Application Domain: Rare SOZ Detection

The raw 4D spatio-temporal fMRI data is processed using Independent component (IC) analysis to get three kinds of ICs: noise, that captures measurement artifacts, resting state networks (RSN) that capture normal brain activity, and seizure onset zone (SOZ) ICs (rare class). fMRI yields between 150 ICs per patient with (<10%) associated with the SOZ. The problem is: given an IC we need to classify if it belongs to SOZ.

TABLE 1
Performance results of SOTA models and CuKPL on Center A (Top-half).
Performance results for all models trained on data from Center A and evaluated on
unseen data from Center B (Bottom-half). MM: Machine marked

| Method | Accuracy | Precision | Sensitivity | F1-score | MM SOZs |
|---|---|---|---|---|---|
| DL-2D CNN | 46.1% | 88.8% | 48.9% | 63.0% | 10 |
| DL-ViT | 34.6% | 85.7% | 36.7% | 51.3% | 13 |
| Knowledge-based system (EPIK) | 75.0% | 92.8% | 79.5% | 85.6% | 43 |
| Supervised-Knowledge-based system | 50.0% | 89.6% | 53.6% | 67.0% | 6 |
| Knowledge and DL (DeepXSOZ) | 84.6% | 93.6% | 89.7% | 91.6% | 18 |
| Standard zero shot (LVM-CLIP) | All ICs classified as Noise | — | — | — | — |
| CuPL | Most ICs classified | — | — | — | — |
| | as Noise, few as RSN | | | | |
| CuPKL GPT-40 | 88.4% | 93.8% | 93.8% | 93.8% | 28 |
| DL-2D CNN Center B | 67.7% | 87.5% | 75.0% | 80.7% | 14 |
| DL-ViT Center B | 12.9% | 57.1% | 14.2% | 22.7% | 6 |
| Knowledge-based system (EPIK) Center B | 52.1% | 85.7% | 58.1% | 68.5% | 27 |
| Supervised-Knowledge-based system, Center B | 83.8% | 89.6% | 92.8% | 91.2% | 21 |
| Knowledge and DL (DeepXSOZ) Center B | 90.3% | 90.3% | 100% | 94.9% | 28 |
| CuPKL GPT-40 Center B | 70.0% | 90.3% | 75.0% | 82.3% | 14 |

Solution: Few key knowledge instances on a SOZ include: a) Single activation within the brain, b) Activation in gray matter, may overlap with white matter, c) Assymetry in hemisphere's activations, etc. This raw knowledge was compiled from literature and further refined by LLM to focus on discriminative knowledge (FIG. 2, top). Each specific knowledge component (K1, K2, . . . , KN) was encoded using the LLM, triggering distinct prompts when the corresponding image aligned with domain knowledge (FIG. 1). These knowledge-specific components, forming the HKETP, are integrated with the refined knowledge relevant to the category of interest. For inference, a spatial IC is given to the knowledge model, generating HKETP. HKETP and refined knowledge is used by LLM to classify the SOZ using its deductive reasoning.

Customized prompt example by CuKPL

Rules of seizure onset zone (SOZ) are as follows:
1. Detection of 1 big red activation.
2. Big red activation has asymmetry in hemisphere.
3. Big red activation has NO similar shape.
Rules of being not SOZ:
1. Lots of small/medium activations detected.
2. Symmetry in activations in both hemispheres.
3. Detection of 1 big red activation has majority overlap in gray matter.
Based on rules, answer in YES/NO if the prompt below belongs to SOZ. If uncertain,
respond NO.
1 big red activation(s) detected! 4 small red activation(s) detected! Big red activation
has Percentage overlap with grey matter: 92.5%, with white matter: 0.0%, The big red
activation has asymmetrical shapes.

### 4. Experimental Setup and Evaluation

Data collected from two centers, A and B, in compliance with IRB protocols. Center A, PCH, Phoenix, has 52 pediatric patients (23 Male, 29 Female, ages 3 months to 18 years) with 5,616 images (2,873 Noise, 2,427 RSN, 316 SOZ), acquired with 3T Philips Ingenuity. Center B, UNC, Chapel hills, has 31 patients (14 Male, 17 Female, ages 2 months to 62 years) with 2,364 images (1,090 Noise, 1,072 RSN, 202 SOZ), acquired using a Siemens MAGNETOM Prisma FIT.

Evaluation: CuKPL was implemented using GPT-40. CuKPL was first compared with ViT and CNN. Knowledge techniques are then evaluated: EPIK, Hunyadi et al. to evaluate the effectiveness of knowledge on rare class classification. The integration of knowledge and DL is then evaluated. Finally, SOTA zero shot image classification models are evaluated using open vocabulary systems. The experiments were run on an Intel Xeon CPU with 8 cores, 93 GB RAM and NVIDIA Quadro RTX 5000 GPU with 16 GB memory. For each method, two evaluations were performed: (1) Training on center A and evaluating on center A with Leave-One-Out Cross-Validation, and (2) Training on center A and testing on unseen center B. The potential effort reduction for the surgical team was also assessed by comparing the ICs that require review by the surgical expert to those that would need manual sorting using machine marked (MM) SOZs.

Results Analysis: SOTA DL techniques like 2D-CNNs and ViT, yielded suboptimal results (see Table 1). Knowledge-based approaches, such as EPIK and supervised knowledge-based systems, offered improvements suggesting that human expert knowledge is valuable than pure data driven learning. Combining knowledge with DL achieved optimal results but remained data-dependent, non-zero-shot, and required extensive, time-consuming training. To address these limitations, the standard zero-shot methods like CLIP and CUPL were evaluated, but neither possessed knowledge of rare medical events. This prompted the development of CuKPL, which generates HKETPs and achieved a 3.8% accuracy and 2.2% F1 score improvement on Center A data over SOTA DeepXSOZ. In single-domain generalization, CuKPL showed strong performance on Center B, with an F1 score of 82.3% and 70% accuracy. The integration of knowledge and DL proved most effective across evaluations. The MM SOZs of 28 show a significant reduction in manual evaluation by neurosurgeons, making them ideal for clinical use.

### 5. Non-Limiting Conclusions

Traditional DL methods underperform with limited data, and purely knowledge-based approaches fall short due to vague domain knowledge. The inventive approach and/or framework, dubbed herein as CuKPL, overcomes these limitations by generating HKETP combined with quantification modules, then feeding them into the LLM for classification. Extensive testing shows that CuKPL outperforms SOTA methods and achieves decent single-domain generalization across various datasets. Additionally, this solution generalizes well to other medical image classification tasks like proliferative diabetic retinopathy grading by leveraging domain knowledge to improve accuracy despite limited data.

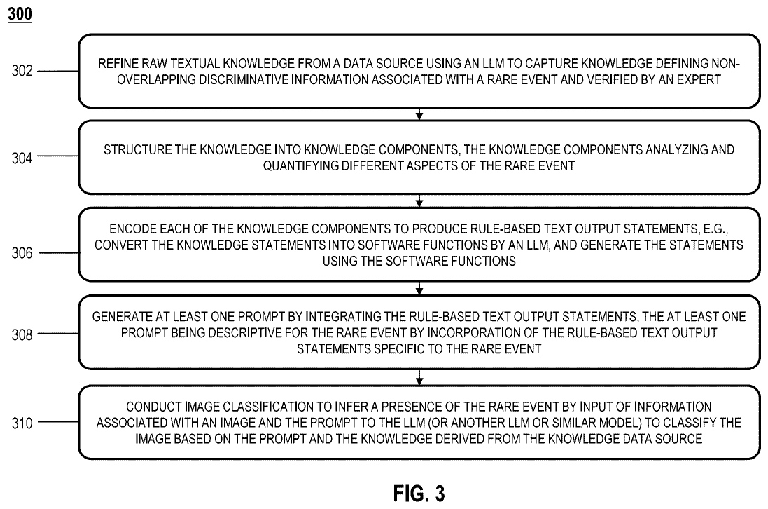

Exemplary Method/Process: Referring to FIG. 3, an example method or process is provided which can be implemented as machine readable instructions and/or via any type or number of processing elements.

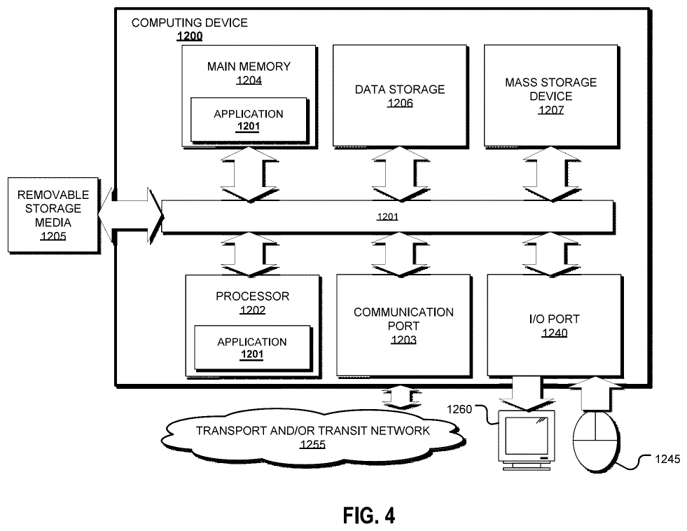

Exemplary Computing Device: Referring to FIG. 4, a computing device 1200 is illustrated which can be configured, via one or more of an application 1211 or computer-executable instructions, to execute functionality described herein. More particularly, in some embodiments, aspects of the methods herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein. Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

The described methods, processes, operations, and associated actions may also be performed in various orders in addition to the order described in this application, in parallel, and/or simultaneously. The described systems are exemplary in nature and may include additional elements and/or omit elements. Furthermore, references to or “one example” of the present disclosure are not intended to be interpreted as excluding the existence of additional embodiments that also incorporate the recited features. It will be understood that when a certain part or process “includes” a certain component or operation, that part or process does not exclude another component or operation. While illustrative examples of the name screening techniques using phonetic embeddings have been described herein including systems, devices, and the like, it is to be understood that various other adaptations and modifications may be made within the spirit and the scope of the examples herein. Additionally, it is appreciated that while specific graphics are shown and described, such graphics are illustrative and exemplary and are not intended to limit the scope of this disclosure.

The foregoing description has been directed to specific examples. It will be apparent, however, that other variations and modifications may be made to the described examples, with the attainment of some or all of their advantages. For instance, it is expressly contemplated that the components and/or elements described herein can be implemented as software being stored on a tangible (non-transitory) computer-readable medium, devices, and memories (e.g., disks/CDs/RAM/EEPROM/etc.) having program instructions executing on a computer, hardware, firmware, or a combination thereof. Further, methods describing the various functions and techniques described herein can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on. In addition, devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example. Instructions, media for conveying such instructions, computing resources for executing them, and other structures for supporting such computing resources are means for providing the functions described in these disclosures. Accordingly, this description is to be taken only by way of example and not to otherwise limit the scope of the examples herein. Therefore, it is the object of the appended claims to cover all such variations and modifications as come within the true spirit and scope of the examples herein.

In addition, the description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

Additional aspects of this disclosure are set out in the independent claims and preferred features are set out in the dependent claims. Features of one aspect may be applied to each aspect alone or in combination with other aspects. In addition, while certain operations in the claims are provided in a particular order, it is appreciated that such order is not required unless the context otherwise indicates.

What is claimed is:

1. A computer-implemented method for zero-shot classification of a rare event in medical imagery, comprising:
accessing raw textual knowledge describing a rare event class from one or more knowledge data sources;
refining, by at least one processor using one or more large language models, the raw textual knowledge to generate refined knowledge comprising non-overlapping discriminative characteristics for the rare event class, wherein the refined knowledge is configured to be verifiable by an expert;
structuring the refined knowledge into a plurality of knowledge components, each knowledge component corresponding to a respective discriminative characteristic and being configured to analyze or quantify an aspect of the rare event class;
for an input medical image, generating, by a knowledge module, image-derived attributes comprising at least one quantified measurement by processing the input medical image to compute at least one quantified measurement characterizing one or more detected regions other image features represented in the input medical image;
encoding the plurality of knowledge components to produce rule-based text output statements based on the image-derived attributes, each rule-based text output statement representing whether a corresponding knowledge component is satisfied;
generating a customized prompt by integrating the rule-based text output statements and at least a portion of the refined knowledge such that the customized prompt integrates the refined knowledge with the rule-based text output statements to provide a structured textual representation of the input medical image for rule-guided classification of the rare event class; and
causing at least one of the one or more large language models to process the customized prompt without the input medical image to produce a classification output indicating whether the input medical image is associated with the rare event class.

2. The computer-implemented method of claim 1, wherein refining the raw textual knowledge comprises generating, using the one or more large language models, a set of discriminative rules for the rare event class that are non-overlapping with one another.

3. The computer-implemented method of claim 1, wherein structuring the refined knowledge comprises defining the plurality of knowledge components as respective knowledge-software components that each implement a corresponding discriminative rule for the rare event class.

4. The computer-implemented method of claim 3, wherein the plurality of knowledge components comprise at least an activation-count component, an anatomical overlap component, and an asymmetry component that respectively evaluate an activation count, an overlap with an anatomical tissue type, and a spatial asymmetry associated with the input medical image.

5. The computer-implemented method of claim 1, wherein encoding the plurality of knowledge components comprises generating, using at least one of the one or more large language models, executable software functions corresponding to the plurality of knowledge components, and executing the software functions to generate the rule-based text output statements.

6. The computer-implemented method of claim 1, wherein generating the image-derived attributes comprises performing image-to-text extraction of the input medical image to generate the structured textual representation such that the structured textual representation includes quantified measurements and excludes pixel data of the input medical image.

7. The computer-implemented method of claim 1, wherein generating the customized prompt comprises combining (i) the refined knowledge as class rules for the rare event class with (ii) the rule-based text output statements as an instance description corresponding to the input medical image.

8. The computer-implemented method of claim 1, wherein the customized prompt further comprises at least one non-class rule indicative of a non-rare-event class, and wherein causing the at least one of the one or more large language models to process the customized prompt comprises directing the at least one of the one or more large language models to classify the input medical image based on satisfaction of the class rules and the at least one non-class rule.

9. The computer-implemented method of claim 1, further comprising receiving feedback from a human verifier regarding at least one of the refined knowledge or the plurality of knowledge components, and updating at least one of the refined knowledge or the plurality of knowledge components based on the feedback.

10. The computer-implemented method of claim 1, wherein causing the at least one of the one or more large language models to process the customized prompt comprises transmitting the customized prompt to a language model that is remote from the knowledge module, without transmitting the input medical image to the language model, thereby preserving patient privacy.

11. A method for zero-shot rare event image classification, comprising:
refining raw textual data from a knowledge data source using a Large Language Model (LLM) to capture knowledge defining non-overlapping discriminative information associated with a rare event and verified by an expert;
structuring the knowledge into knowledge components, the knowledge components analyzing and quantifying different aspects of the rare event;
encoding each of the knowledge components to produce rule-based text output statements; and
generating at least one prompt by integrating the rule-based text output statements, the at least one prompt being descriptive for the rare event by incorporation of the rule-based text output statements specific to the rare event.

12. The method of claim 11, further comprising:
conducting image classification to infer a presence of the rare event by inputting information associated with an image and the prompt to the LLM to classify the image based on the prompt and the knowledge derived from the knowledge data source.

13. The method of claim 11, wherein the at least one prompt includes a degree of satisfiability based on a given input image.

14. The method of claim 11, wherein the knowledge data source includes domain-specific natural language information from an expert known to be true and associated with the rare event.

15. The method of claim 11, wherein the at least one prompt couples knowledge on the rare event from human experts with Large language models (LLM) to enable zero-shot rare event classification.

16. The method of claim 11, wherein the raw textual data includes general descriptions associated with various characteristics of the rare event.

17. The method of claim 12, further comprising conducting image to text extraction of the image to preserve data privacy by omitting input of the image itself to the LLM.

18. The method of claim 11, further comprising:
encoding the knowledge components into software functions code by an LLM,
generating, using the software functions, the rule-based text output statements; and
concatenating the rule-based text output statements with the prompt,
wherein the prompt is customized for the rare event and is configured for input to any LLM for inference or prediction of the rare event.

19. A system for zero-shot classification of a rare event in medical imagery, comprising:
one or more processors; and
non-transitory memory storing instructions that, when executed by the one or more processors, cause the system to:
refine raw textual knowledge using one or more large language models to generate refined knowledge comprising non-overlapping discriminative knowledge specific to a rare event class;
structure the refined knowledge into knowledge components, and encode the knowledge components, using at least one of the one or more large language models, to generate rule-based text outputs corresponding to the knowledge components;
for an input medical image, generate targeted prompt text for respective ones of the knowledge components;
integrate the targeted prompt text to generate a human knowledge embodied textual prompt (HKETP); and
generate a customized prompt for rare event classification by combining the HKETP with at least a portion of the refined knowledge.

20. The system of claim 19, the non-transitory memory storing further instructions that, when executed by the one or more processors, cause the system to:
cause at least one of the one or more large language models to process the customized prompt to generate a classification output for the input medical image.
